(19) United States
(12) Patent Application Publication
(10) Pub. No.: US 2018/0177907 A1
(43) Pub. Date: Jun. 28, 2018

(54) FRAGRANCE LAMP AND USING METHOD

(71) Applicant: Zhongshan Invitop Electrical Co.,Ltd., Zhongshan (CN)

(72) Inventor: Yisheng Li, Zhongshan (CN)

(73) Assignee: Zhongshan Invitop Electrical Co.,Ltd.

(21) Appl. No.: 15/451,398

(22) Filed: Mar. 6, 2017

(30) Foreign Application Priority Data
Dec. 22, 2016 (CN) 201611200287.8

(51) Int. Cl. A61L 9/14; B05B 17/06; F21S 10/04; F21V 23/04; F21S 9/02; F21V 21/15

(52) U.S. Cl. CPC A61L 9/14; B05B 17/0607; F21S 10/046; A61L 2209/132; F21S 9/02; F21V 21/15; A61L 2209/12; F21V 23/0435

(57) ABSTRACT

The present invention discloses a fragrance lamp and a using method. The fragrance lamp comprises a casing, a water tank and a base, wherein the casing is composed of a mist outlet, a flame piece and a supporting column; the casing is buckled on the upper portion of the water tank; the water tank is provided with a mist guiding cavity and a magnetic field cavity through a water tank cover; the water tank is provided with a spotlight module and a magnetic induction device through a fan-shaped cover; a magnetic induction coil is installed on the magnetic induction device, the bottom of the water tank is provided with an ultrasonic device, the bottom end of the water tank is connected with the base, a direct current (DC) socket is buckled on the base, a circuit board and a power supply key are disposed in the base, and the circuit board is in signal connection with a remote control device through a wireless transmission module. The spotlight module is controlled through the remote control device to gather light to a flame piece, a mist spraying effect is generated through the mist guiding cavity, the flame piece swings regularly under the effect of the magnetic induction device, a personalized light effect is formed, and the fragrance lamp is novel in structure and easy to use in function.

### CROSS-REFERENCE TO RELATED APPLICATIONS

This application claims priority to Chinese Patent Application No. 201611200287.8 with a filing date of Dec. 22, 2016. The content of the aforementioned application, including any intervening amendments thereto, are incorporated herein by reference.

### TECHNICAL FIELD

The present invention relates to the technical field of environment-friendly light-emitting devices, and in particular to a fragrance lamp and a using method.

### BACKGROUND OF THE PRESENT INVENTION

Visual effects of light of an aromatherapy machine directly affect users' emotions; and particularly when users want to relieve pressure by means of the aromatherapy machine after being deeply subjected to work pressure, unreasonable light design has a more severe influence. At present, there are various aromatherapy machines on the market, and the aromatherapy machines have a certain aromatherapy function and visual effect. Water in the aromatherapy machines becomes mist through ultrasonic vibration and then is blown out through an air outlet, and lamps are universally designed at the bottoms of water tanks or directly installed on circuit boards.

These devices have a certain visual light effects; however, due to the fact that the devices are simple in structure, the influence on circuits caused by mist condensation water drops cannot be overcome, and thus creative design cannot be further performed on the visual light effects. For consumers suffering from poor sleep, when they need to improve sleep quality by means of the aromatherapy machines, if the light effect is not designed appropriately, e.g., if the light is bright and dazzling, it will lead to a poorer influence.

### SUMMARY OF PRESENT INVENTION

The objective of the present invention is to provide a fragrance lamp and a using method, so as to solve the problems mentioned in the background art.

In order to achieve the aforementioned objective, the present invention provides the following technical solution: a fragrance lamp comprising a casing, a water tank and a base, wherein the casing and the base are provided with limiting devices; the base is provided with a power supply key for turning on the fragrance lamp; the casing is composed of a mist outlet, a flame piece and a supporting column; a fixing column is arranged in the flame piece, a magnet is arranged at the bottom of the flame piece, the flame piece is fixed to the supporting column through the fixing column, and the casing is buckled on the upper portion of the water tank; the top end of the water tank is provided with a water tank cover, and a water tank inner cover is buckled on the lower portion of the water tank cover; a mist guiding pipe is arranged in the water tank cover and the water tank inner cover, the water tank is provided with a fan-shaped cover, the fan-shaped cover is tightly buckled on a fan-shaped opening of the water tank, a spotlight module is disposed in the fan-shaped cover, a spotlight is fixed to the spotlight module, a magnetic induction device is installed in the water tank, the bottom of the water tank is provided with an ultrasonic device, and a induction coil is installed at the bottom of the water tank; the bottom end of the water tank is connected with the base, a direct current (DC) socket is buckled on the base, a circuit board is disposed in the base, an outer ring of the circuit board is provided with four screws, the circuit board is fixed into the base through the screws, a direct current fan is disposed below the circuit board and is pressed in the base through the circuit board, the inner side of the bottom end of the base is provided with a clamping groove, a key board is clamped in the clamping groove, the outer side of the bottom end of the base is provided with a specific round hole, a power supply key is installed on the specific round hole, and the key board fixes the power supply key; and the key board, the direct current fan, the ultrasonic device, the magnetic induction device and the spotlight module are electrically connected with the circuit board.

Preferably, when the water tank inner cover is installed in the water tank cover to be combined and matched with the water tank, two relatively independent cavities are formed; one cavity is a mist-free and water-free magnetic field cavity, and the other cavity is a mist guiding cavity.

Preferably, the circuit board is in signal matching connection with a remote control device through a wireless transmission module, and the remote control device is provided with a 1H/2H/3H timing indicating lamp, a fan switch key, a timing key, a mist amount adjusting key and a light switch key.

Preferably, the method for using the fragrance lamp comprises the following steps:

Step A of assembling the fragrance lamp well, placing the fragrance lamp at an appropriate position, and pressing a switch of the power supply key;

Step B of matching the remote control device with the circuit board through the wireless transmission module, and displaying a matching state through the indicating lamp;

Step C of pressing the light switch key on the remote control device, controlling the spotlight on the spotlight module to emit light, and generating a spotlight effect by the flame piece;

Step D of pressing the mist amount adjusting key on the remote control device to control the water tank to generate mist in the mist guiding cavity, when the flame piece swings back and forth along with the cutting of the magnetic induction device;

Step E of pressing the fan switch key on the remote control device, blowing mist generated in the mist guiding cavity to the mist outlet of the casing through the direct current fan;

Step F of setting 1H/2H/3H timing work of the fragrance lamp through the timing key on the remote control device;

Step G of switching off the switch of the power supply key after Step F is executed, and cutting off a mode of wireless connection between the fragrance lamp and the remote control device.

Compared with the prior art, the present invention has the beneficial effects that the spotlight module is installed at the top of the water tank to overcome interference caused by water and mist; the flame piece swings regularly at the top of the casing under the magnetic force effect of the magnetic induction device; light of the spotlight shines the flame piece, and through the swing of the flame piece, the light effect is like lit fragrance; the water tank forms two relatively independent cavities together with the water tank cover and the water tank inner cover; one cavity is a mist-free and water-free magnetic field cavity, and the other cavity is a mist guiding cavity. The light control, mist amount regulation and timing in the present invention are performed through remote control, and the intelligence degree is high.

### DESCRIPTION OF THE DRAWINGS

FIG. 1 is an exploded view of an integrated structure according to Embodiment 1 of the present invention; and

FIG. 2 is an exploded view of an integrated structure according to Embodiment 2 of the present invention.

### DETAILED DESCRIPTION OF PREFERRED EMBODIMENTS

The technical solutions in embodiments of the present invention will be described clearly and completely below with reference to accompanying drawings in the embodiments of the present invention. Obviously, the described embodiments are merely part of embodiments of the present invention rather than all embodiments. Based on the embodiments of the present invention, all other embodiments obtained by those skilled in the art without creative labor shall belong to the protective scope of the present invention.

### Embodiment 1

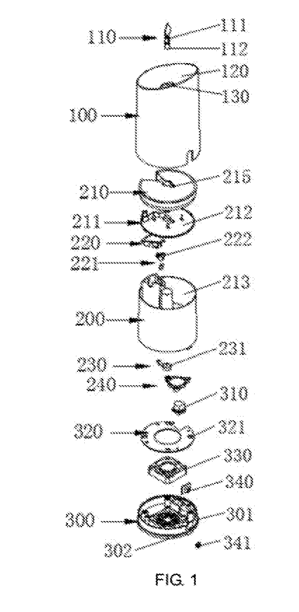

Referring to FIG. 1, the present invention provides a technical solution: a fragrance lamp comprising a casing 100, a water tank 200 and a base 300, wherein the casing 100 is composed of a mist outlet, a flame piece 110 and a supporting column 130; a fixing column 111 is arranged in the flame piece 110, a magnet 112 is arranged at the bottom of the flame piece 110, and the flame piece 110 is fixed to the supporting column 130 through the fixing column 111; when an aromatherapy machine works, the flame piece 110 can be remotely set to swing or be static; the casing 100 is buckled on the upper portion of the water tank 200; the top end of the water tank 200 is provided with a water tank cover 210, and a water tank inner cover 211 is buckled on the lower portion of the water tank cover 210; an independent air duct is formed in the water tank inner cover 211; when the water tank inner cover 211 is installed in the water tank cover 210 to be combined and matched with the water tank 200, two relatively independent cavities are formed; one cavity is a mist-free and water-free magnetic field cavity 212, and the other cavity is a mist guiding cavity 213; a mist guiding pipe 215 is arranged in the water tank cover 210 and the water tank inner cover 211, the water tank 200 is provided with a fan-shaped cover 220, the fan-shaped cover 220 is tightly buckled on a fan-shaped opening of a water tank 200, a spotlight module 221 is disposed in the fan-shaped cover 220, a spotlight 222 is fixed to the spotlight module 221 and projected to the flame piece 110, and the flame piece 110 swings under the effect of magnetic force to form an effect of the fragrance lamp; a magnetic induction device 230 is installed in the water tank 200; in order that the magnetic induction device 230 can be electrified to generate a magnetic field and is not subjected influences of mist, water and structure wall thickness, a magnetic induction coil 231 is installed inside the back of the water tank 200; and the flame piece 110 is very close to the magnetic induction coil 231, so that the magnet 112 can be at the position of the highest magnetic field generated by the magnetic induction coil 231.

In this embodiment, the bottom of the water tank 200 is provided with an ultrasonic device 240, the bottom end of the water tank 200 is connected with the base 300, a direct current (DC) socket 310 is buckled on the base 300, a circuit board 320 is disposed in the base 300, an outer ring of the circuit board 320 is provided with four screws 321, the circuit board 320 is fixed into the base 300 through the screws 321, a direct current fan 330 is disposed below the circuit board 320, the circuit board 320 presses the direct current fan 330 into the base 300, the inner side of the bottom end of the base 300 is provided with a clamping groove 301, a key board 340 is clamped in the clamping groove 301, the outer side of the bottom end of the base 300 is provided with a specific round hole 302, a power supply key 341 is installed on the specific round hole 302, and the key board 340 fixes the power supply key 341; the light control, mist amount regulation and timing are performed through remote control, and a remote control device is provided with a 1H/2H/3H timing indicating lamp, a timing key, a mist amount adjusting key and a light switch key. In order to gather light of the fragrance lamp to the flame piece 110 well, a blade of the flame piece 110 is designed into a shape with the periphery recessed towards the middle, and the blade swings regularly through a magnetic field generated by the cutting of the magnetic induction device 230 by the magnet 112 arranged at the bottom of the flame piece 110. The internal structural design of the water tank inner cover 211 and the water tank cover 210 can guarantee that mist generated by the water tank 200 can only be blown to a mist outlet 120 of the casing 100 from the mist guiding pipe 215 and is not leaked from other places, and it can also be guaranteed that the magnetic induction cutting by the magnet 112 arranged at the bottom of the flame piece 110 is not disturbed; it can be guaranteed that the flame piece 110 swings without hindrance and does not fall in the process of swing, casing movement and product transportation, and the fixing position of the lamp is close to the flame piece 110. Such a fixing method enables light to be gathered at the center of the blade of the flame piece 110 more accurately, the spotlight 222 is clamped on a lamp holder and maintains a certain angle, the lamp holder is flexible in structure, it can be guaranteed that the spotlight 222 is convenient to install, and it can also be guaranteed that the spotlight 222 is not taken out easily; the casing 100 and the base 300 have limitations, and thus the accuracy of a light projection direction is further guaranteed.

The using method according to the present invention comprises the following steps:

Step A of assembling the fragrance lamp well, placing the fragrance lamp at an appropriate position, and pressing a switch of the power supply key 341;

Step B of matching the remote control device with the circuit board 320 through the wireless transmission module, and displaying a matching state through the indicating lamp;

Step C of pressing the light switch key on the remote control device, controlling the spotlight 222 on the spotlight module 221 to emit light, and generating a spotlight effect by the flame piece 110;

Step D of pressing the mist amount adjusting key on the remote control device to control the water tank 200 to generate mist in the mist guiding cavity 213, when the flame piece 110 swings back and forth along with the cutting of the magnetic induction device 230;

Step E of pressing a fan switch key on the remote control device, blowing mist generated in the mist guiding cavity 213 to the mist outlet of the casing 100 through the direct current fan 330;

Step F of setting 1H/2H/3H timing work of the fragrance lamp through the timing key on the remote control device;

Step G of switching off the switch of the power supply key 341 after Step F is executed, and cutting off a mode of wireless connection between the fragrance lamp and the remote control device.

In this embodiment, the remote control device is provided with the 1H/2H/3H timing indicating lamp, the fan switch key, the timing key, the mist amount adjusting key and the light switch key. In order to gather light to the flame piece 110 well, the blade of the flame piece 110 is designed into a shape with the periphery recessed towards the middle, and the blade swings regularly through a magnetic field generated by the cutting of the magnetic induction device 230 by the magnet 112 arranged at the bottom of the flame piece 110. The internal structural design of the water tank inner cover 211 and the water tank cover 210 can guarantee that mist generated by the water tank 200 can only be blown to the mist outlet 120 of the casing 100 from the mist guiding pipe 215 and is not leaked from other places, and it can also be guaranteed that the magnetic induction cutting by the magnet 112 arranged at the bottom of the flame piece 110 is not disturbed; it can be guaranteed that the flame piece 110 swings without hindrance and does not fall in the process of swing, casing movement and product transportation, and the fixing position of the lamp is close to the flame piece 110. Such a fixing method enables light to be gathered at the center of the blade of the flame piece 110 more accurately, the spotlight 222 is clamped on a lamp holder and maintains a certain angle, the lamp holder is flexible in structure, so it can be guaranteed that the spotlight 222 is convenient to install, and it can also be guaranteed that the spotlight 222 is not taken out easily; the casing 100 and the base 300 have limitations, and thus the accuracy of a light projection direction is further guaranteed. The spotlight module 221 is installed at the top of the water tank to overcome interference caused by water and mist; the flame piece 110 swings regularly at the top of the casing under the magnetic force effect of the magnetic induction device 230; light of the spotlight 222 shines the flame piece 110, and through the swing of the flame piece 110, the light effect is like lit fragrance; the water tank 200 forms two relatively independent cavities together with the water tank cover 210 and the water tank inner cover 211; one cavity is a mist-free and water-free magnetic field cavity 212, and the other cavity is a mist guiding cavity 213. The fragrance lamp has a personalized light effect and is novel in structure and easy to use in function.

### Embodiment 2

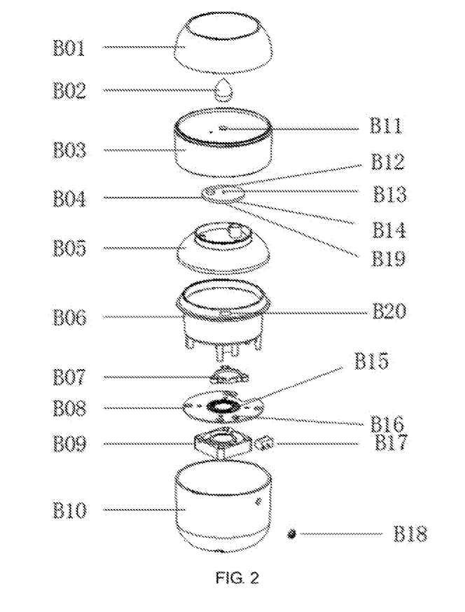

Referring to FIG. 2, the present invention provides a technical solution: a fragrance lamp comprising an upper cover component B03, a water tank B06 and a base B10, wherein the upper cover component B03 is composed of a transparent cover B01, an upper cover B03 and a water tank cover B05; the transparent cover B01 is a transparent plastic part with the outer surface provided with a ground surface, the transparent cover B01 is buckled on the upper portion of the upper cover B03 in a buckle mode, the water tank cover B05 is buckled on the lower portion of the upper cover B03, the upper cover B03 is provided with a mist outlet B11, a decorating lamp B02 is fixed to the mist outlet B11 of the upper cover B03 in a buckle mode, the water tank cover B05 is provided with a battery module B04, and the battery module B04 is fixed inside the top of the water tank cover B05 in a screw mode; the battery module B04 is provided with an electronic control board B19, a storage type battery B12, a lamp B13 and a wireless charging receiver B14; the upper cover B03 is buckled on the upper portion of the water tank B06 (the buckling here is movable to facilitate use by users); the cambered surface of the upper portion of the water tank B06 is provided with an air outlet B20; the bottom of the water tank B06 is provided with an ultrasonic device B07 and a circuit board B08; the circuit board B08 is provided with a wireless charging transmitter B15 and a switch key B16; the bottom end of the water tank B06 is connected with the base B10; a DC socket B17 and a direct current fan B09 are buckled on the base B10, and a power supply key B18 is fixed through a switch key B16 on the circuit board B08.

The using method according to this embodiment comprises the following steps:

Step A of assembling the fragrance lamp well, placing the fragrance lamp at an appropriate position, and pressing a switch of the power supply key B18;

Step B of matching the remote control device with the circuit board B08 through the wireless transmission module, and displaying a matching state through the indicating lamp;

Step C of pressing the light switch key on the remote control device, controlling the lamp B13 on the battery module B04 to emit light;

Step D of pressing the mist amount adjusting key on the remote control device after Step C is executed, controlling the ultrasonic device B07 of the water tank B06 to generate mist, and at this time also starting the direct current fan B09;

Step E of setting 1H/2H/3H timing work of the fragrance lamp through the timing key on the remote control device;

Step F of switching off the switch of the power supply key B18 after Step E is executed, and cutting off a mode of wireless connection between the fragrance lamp and the remote control device.

In this embodiment, mist generated by the ultrasonic device is sprayed out of the mist outlet where the decorating lamp is buckled, light is projected on the mist through the decorating lamp, the mist moves upwards, so that it is like that a beam of burning flame is formed, and the effect seen from outside the transparent cover is like a campfire. The remote control device is provided with the 1H/2H/3H timing indicating lamp, the timing key, the mist amount adjusting key and the light switch key; to make the light effect look like the burning campfire, the decorating lamp B02 is designed ingeniously; besides, the surfaces of the decorating lamp B02 and the transparent cover B01 are ground surfaces, the decorating lamp B02 can be fixed and guarantee that mist is discharged, and mist condensation water at this position does not have an influence on an internal circuit; for convenience in use by users, a circuit portion in the upper cover component B03 is connected with a circuit portion of a base plate of the water tank B06 in a wireless charging mode; for improvement of production efficiency, the circuit board B08 is fixedly installed at the bottom of the water tank; and the fragrance lamp has a personalized light effect and is novel in structure and easy to use in function.

Though the embodiments of the present invention have been illustrated and described, those skilled in the art can perform various variations, modifications and replacements on these embodiments without departing from the principle and the spirit of the present invention, and the range of the present invention is defined by the appended claims and equivalents thereof.

I claim:

1. A fragrance lamp, comprising a casing (100), a water tank (200) and a base (300), wherein the casing (100) and the base (300) are provided with limiting devices; the base (300) is provided with a power supply key (341) for turning on the fragrance lamp; the casing (100) is composed of a mist outlet (120), a flame piece (110) and a supporting column (130); a fixing column (111) is arranged in the flame piece (110), a magnet (112) is arranged at the bottom of the flame piece (110), and the flame piece (110) is fixed to the supporting column (130) through the fixing column (111); the casing (100) is buckled on the upper portion of the water tank (200); the top end of the water tank (200) is provided with a water tank cover (210), and a water tank inner cover (211) is buckled on the lower portion of the water tank cover (210); a mist guiding pipe (215) is arranged in the water tank cover (210) and the water tank inner cover (211), the water tank (200) is provided with a fan-shaped cover (220), and the fan-shaped cover (220) is tightly buckled on a fan-shaped opening of the water tank (200); a spotlight module (221) is disposed in the fan-shaped cover (220), a spotlight (222) is fixed to the spotlight module (221), and a magnetic induction device (230) is installed in the water tank (200); the bottom of the water tank (200) is provided with an ultrasonic device (240), and a magnetic induction coil (231) is installed at the bottom of the water tank (200); the bottom end of the water tank (200) is connected with the base (300), a direct current (DC) socket (310) is buckled on the base (300), a circuit board (320) is disposed in the base (300), an outer ring of the circuit board (320) is provided with four screws (321), the circuit board (320) is fixed into the base (300) through the screws (321), a direct current fan (330) is disposed below the circuit board (320) and is pressed in the base (300) through the circuit board (320), the inner side of the bottom end of the base (300) is provided with a clamping groove (301), a key board (340) is clamped in the clamping groove (301), the outer side of the bottom end of the base (300) is provided with a specific round hole (302), a power supply key (341) is installed on the specific round hole (302), and the key board (340) fixes the power supply key (341); and the key board (340), the direct current fan (330), the ultrasonic device (240), the magnetic induction device (230) and the spotlight module (221) are electrically connected with the circuit board (320).

2. The fragrance lamp according to claim 1, wherein when the water tank inner cover (211) is installed in the water tank cover (210) to be combined and matched with the water tank (200), two relatively independent cavities are formed; one cavity is a mist-free and water-free magnetic field cavity (212), and the other cavity is a mist guiding cavity (213).

3. The fragrance lamp according to claim 1, wherein the circuit board (320) is in signal matching connection with a remote control device through a wireless transmission module, and the remote control device is provided with a 1H/2H/3H timing indicating lamp, a fan switch key, a timing key, a mist amount adjusting key and a light switch key.

4. The fragrance lamp according to claim 1, wherein a method for using the fragrance lamp comprises the following steps:
Step A of assembling the fragrance lamp well, placing the fragrance lamp at an appropriate position, and pressing a switch of the power supply key (341);
Step B of matching the remote control device with the circuit board (320) through the wireless transmission module, and displaying a matching state through the indicating lamp;
Step C of pressing the light switch key on the remote control device, controlling the spotlight (222) on the spotlight module (221) to emit light, and generating a spotlight effect by the flame piece (110);
Step D of pressing the mist amount adjusting key on the remote control device to control the water tank (200) to generate mist in the mist guiding cavity (213), when the flame piece (110) swings back and forth along with the cutting of the magnetic induction device (230);
Step E of pressing the fan switch key on the remote control device, blowing mist generated in the mist guiding cavity (213) to the mist outlet of the casing (100) through the direct current fan (330);
Step F of setting 1H/2H/3H timing work of the fragrance lamp through the timing key on the remote control device;
Step G of switching off the switch of the power supply key (341) after Step F is executed, and cutting off a mode of wireless connection between the fragrance lamp and the remote control device.
